(19) United States
(12) Patent Application Publication
(10) Pub. No.: US 2005/0038005 A1
(43) Pub. Date: Feb. 17, 2005

(54) Method of intermittent administration of a pharmaceutical for the treatment of conditions associated with a female's menstrual cycle

(76) Inventors: Roger M. Boissonneault, Long Valley, NJ (US); Tina M. deVries, Long Valley, NJ (US); Herman Ellman, Boonton Township, NJ (US); Kathryn L. MacFarlane, Morristown, NJ (US); Kelly F. Smith, Mountain Lakes, NJ (US)

Correspondence Address: FITZPATRICK CELLA HARPER & SCINTO, 30 ROCKEFELLER PLAZA, NEW YORK, NY, 10112, US

(21) Appl. No.: 10/819,764

(22) Filed: Apr. 7, 2004

Related U.S. Application Data
(63) Continuation-in-part of application No. 10/762,263, filed on Jan. 23, 2004.
Application No. 10/762,263 is a continuation-in-part of application No. 10/639,891, filed on Aug. 12, 2003, now abandoned.

(51) Int. Cl. A61K 31/56

(52) U.S. Cl. 514/170

(57) ABSTRACT

The present invention is a method of non-continuous administration of a pharmaceutical to a female for a condition associated with the female's menstrual cycle, comprising ascertaining a number of days in the female's menstrual cycle, and then administering a daily first dosage, starting on the first or the second day of the menstrual cycle and continuing the daily administration for a first dosage period. Next administering a daily second dosage, starting on the day after the last daily first dosage was administered and continuing the daily administration of the second dosage for a second dosage period. Wherein either the first dosage is a placebo and the second dosage is the pharmaceutical, or the first dosage is the pharmaceutical and the second dosage is a placebo. Both the first dosages and the second dosages being contained in a single package.

### REFERENCE TO RELATED APPLICATION

This application is a continuation-in-part of U.S. patent application Ser. No. 10/762,263, filed Jan. 23, 2004, which is a continuation in-part of U.S. patent application Ser. No. 10/639,891, filed Aug. 12, 2003.

### BACKGROUND OF THE INVENTION

1. Field of the Invention

This invention is directed to a method of intermittent administration of a pharmaceutical to a woman for the treatment of cyclical physical and, or, psychological symptoms that are associated with the menstrual cycle. The invention is also directed to packaging used in the method of administration.

2. Related Background Art

Any number of conditions in women occur intermittently in association with the menstrual cycle. These conditions include, for example, migraine headaches, exacerbation of pain due to endometriosis, and PMDD. A detailed discussion of an example follows: Each month, for a few days prior to the onset of menstruation, many millions of women experience menstrual cycle physical and psychological symptoms, which if found to be repetitive are generally called premenstrual syndrome (PMS). Symptoms of PMS commonly include changes in mood and appetite, which may include becoming saddened, tearful, irritable, angry, anxious, feeling hopelessness or having carbohydrate cravings. In addition, these women suffering from PMS commonly have cyclical physical symptoms, which may include breast tenderness, swelling, headaches, joint or muscle pain, bloating or weight gain.

A severe, predominantly psychological form of the more general PMS is known as premenstrual dysphoric disorder (PMDD), in which general symptoms of PMS markedly interfere with social activities and relationships with others, including at home or work. The Diagnostic and Statistical Manual of Mental Disorders, 4th Edition. Previously PMDD was known as late luteal phase dysphoric disorder (LLPDD). The Diagnostic and Statistical Manual of Mental Disorders, 3rd Edition. PMS, PMDD and LLPDD are used herein interchangeably.

There have been numerous suggestions made about the cause of PMS. For example, some hypothesized that it was caused by a uterine toxin. Others suggested its cause was over consumption of sweets, which was presumably followed by excessive insulin secretion, hypoglycemia, and inadequate brain glucose and resulted in the often observed depression and anxiety. It has also been postulated that the behavioral symptoms result from the tissue edema often observed and that the psychological changes result from feelings of loss or the social complexities generated by the discomforts of menstruation. Some have speculated that progesterone metabolites are implicated.

However, none of these theories has been substantiated: PMS/PMDD can persist after hysterectomy and, hence, uterine toxins cannot be its cause; the hyperinsulinism of PMS/PMDD is not associated with low blood glucose levels, and is probably the consequence of a behavioral aberration (i.e., the tendency of premenstrual women to choose high-carbohydrate diets, which potentiate insulin secretion)—rather than the cause; the mood and appetitive changes of PMS/PMDD are poorly correlated with the tissue swelling; and subhuman primates who are presumably exempt from the psychodynamic or social complexities of human life, also exhibit characteristic behavioral changes premenstrually.

There have been many treatments suggested for overcoming or reducing the symptoms of PMS/PMDD. These include carbohydrate-free diets, reducing salt, alcohol and caffeine intake, vitamin supplements, ovarian hormones, detoxifying agents, irradiation of the ovaries and pituitary, use of diuretics, use of antimineralocorticoids (e.g. aldactone) and use of oral contraceptives.

Calcium has been found to be an effective treatment for alleviating some of the symptoms associated with PMS. It has been especially effective in treating pain and water retention. S. Thys-jacobs, S. Ceccarelli, A. Beirman, H. Weisman, M. A. Cohen and J. Alvir, Calcium supplementation in premenstrual syndrome: A randomized crossover trial, J. Gen. Int. Med. 4:183-189, (1989).

Magnesium supplementation has been shown to relieve some symptoms of PMS, particularly pain and mood fluctuations. F. Fancchinetti, P. Borella, G. Sances, L. Fioroni, R. Nappi and A. R. Genazzani, Oral Magnesium Successfully Relieves Premenstrual Mood Changes, Obstetrics & Gynecology, 78:177-181, (1991).

U.S. Pat. No. 5,354,743 discloses that some women suffering from symptoms associated with PMS have lower levels of 25 hydroxyvitamin D compared to asymptomatic women. Thus, suggesting that vitamin D could be used to alleviate some of the symptoms associated with PMS. Furthermore, vitamin D may be useful in combination with calcium and magnesium supplementation since it is known that vitamin D increases the absorption of calcium and magnesium in the human body and is also involved with the transportation of calcium through cell membranes.

Diuretics have been used to relieve the symptoms related to water retention such as bloating, swelling, painful breasts, cramps and tension. Common diuretics are pamabrom, ammonium chloride, hydrochlorothiazide, and spironolactone.

Nonprescription pharmacological compositions have also been used. These compositions generally contain an analgesic, such as aspirin, acetaminophen or ibuprophen, for the treatment of back pain, headache pain and abdominal cramping. Sometimes these compositions will also contain an antispasmodic to help treat the abdominal cramping.

U.S. Pat. No. 4,897,411 discloses the use of clonidine to treat the symptoms associated with PMS. Clonidine is a centrally acting antihypertensive agent.

U.S. Pat. No. 4,971,998 discloses that administration of d-fenfluramine and fluoxetine, which selectively enhances serotonin-mediated neurotransmission, are useful to treat PMS and LLPDD. Agents or drugs useful in enhancing serotonin-mediated neurotransmission, or the effect of serotonin within the brain synapses, are referred to as serotoninergic drugs and include (1) drugs which act to increase the quantity of serotonin present within the synapses and (2) drugs which act to enhance the effects of serotonin present with brain synapses, generally by activating post-synaptic serotonin receptors.

The neurotransmitter serotonin (5-hydroxytryptamine or 5-HT) is 3-(beta-aminoethyl)-5-hydroxyindole. It stimulates or inhibits a variety of smooth muscles and nerves and, among others, has effects on secretion by both exocrine and endocrine glands and on functioning of the respiratory, cardiovascular and central nervous systems. Within the central nervous system (CNS), serotonin serves as a neurotransmitter in the brain and spinal cord, where it is the chemical transmitter of neurons referred to as tryptaminergic or serotoninergic neurons. These neurons are involved in control of sleep, appetite, nutrient selection, blood pressure, mood, endocrine secretion, aggressivity and numerous other sensitivities to external stimuli.

Administration of fluoxetine, which suppresses reuptake of serotonin and, thus, increases the quantity of serotonin available at brain synapses, has been shown to ameliorate the symptoms of PMS/PMDD. Steiner et al., New England Journal of Medicine, 232:1529-34 (1995). Intermittent administration of fluoxetine for approximately fourteen days prior to the start of menses has also been shown to be efficacious in treating the symptoms of PMS. Cohen et al., Obstet & Gynecol., 100: 435-44 (2000). This suggests that other serotonin reuptake inhibitors would likewise be efficacious in the treatment of the symptoms associated with PMS.

Frequently patients start administration of fluoxetine about 14 days prior to menstruation. Since menstruation generally occurs in 28 day cycles, a good way to determine when to start the administration is to wait 14 days after the prior menstruation cycle has begun. However, this strategy causes difficulty in compliance because it requires the patient keep track of when her cycle has begun and then remember to start taking her medication 14 days later.

The symptoms associated with PMS can be treated with intermittent dosing, which may lessen the likelihood of side effects. Specifically, intermittent dosing may be less likely to cause reduced libido, insomnia and/or anxiety.

A menstrual cycle's length can range between 24 to 33 days in >95% of women with regular cycles, with 28 days being the average length. Thus, any method of treatment for the symptoms of PMS will have to contend with the fact that a women's menstrual cycle can vary in length.

A method of intermittent administration of a pharmaceutical to a woman for the treatment of cyclical physical and, or, psychological symptoms that are associated with the luteal phase of the menstrual cycle commonly referred to as premenstrual syndrome would be highly desirable. In addition, a method of packaging the pharmaceutical to assist the patient in following this method of administration, and thereby, remaining compliant with the treatment would be advantageous.

### SUMMARY OF THE INVENTION

The present invention is a method of non-continuous administration of a pharmaceutical to a human female for a condition associated with the female's menstrual cycle.

In a first embodiment, the method of the invention comprises the steps of:
a) ascertaining a number of days in the female's menstrual cycle;
b) orally administering a daily first dosage, starting on the first day of the female's menstrual cycle and continuing the daily oral administration of the first dosage for a first dosage period; and
c) orally administering a daily second dosage, starting on the day after the last daily first dosage was administered and continuing the daily oral administration of the second dosage for a second dosage period or until the onset of menses. In a first embodiment, where the first dosage is a placebo and the second dosage is the pharmaceutical, the second dosage period is a selected number of days to administer the pharmaceutical for effective treatment of the condition and the first dosage period is determined by subtracting the selected number of days from the number of days in the female's menstrual cycle. In a second embodiment, where the first dosage is the pharmaceutical and the second dosage is a placebo, the first dosage period is a selected number of days to administer the pharmaceutical for effective treatment of the condition and the second dosage period is determined by subtracting the selected number of days from the number of days in the female's menstrual cycle. In addition, the method of this invention requires that the first dosage and the second dosage administered during the menstrual cycle are selected from a single package.

In yet another embodiment of the invention, the oral administration of the daily first dosage begins on the second day of the menstrual cycle. Employing this embodiment of the invention provides that the oral administration of the daily second dosage may be completed on the first day of the menstrual cycle. This embodiment of the method of the invention may be more convenient for the user because menstruation can start at any time of the day and therefore it may be easier to be certain of administering the appropriate daily dosage on the second day following the start of menstruation. On the other hand, it may be equally convenient to simply have the user take both the second dosage from the package in use and the first dosage from a new package when the onset of menses occurs on a day in which the user has already administered a daily second dosage.

Optionally, when the second dosage is a pharmaceutical, it may be preferable upon completion of the second dosage period to continue the daily oral administration of the second daily dosage, i.e., the pharmaceutical, on a daily basis until either, the first of, exhaustion of the pharmaceutical in the package or the start of menses. This option may be useful in a month where the human female's menstrual cycle exceeds the ascertained number of days in the female's menstrual cycle.

The present invention also contemplates a package for delivering a non-continuous administration of a pharmaceutical to a human female for a condition associated with the female's menstrual cycle. The package comprises an allotment of daily placebo dosages, an allotment of daily pharmaceutical dosages and a means of determining how many daily placebo dosages must be administered during the female's menstrual cycle.

The present invention will assist the patient in complying with the treatment. It is known that patients become more readily acclimated to a daily dosage regimen than to an intermittent dosing regimen. Through the use of placebo dosages in conjunction with the pharmaceutical dosages, the patient will be administering a dosage every day and thus, this routine will become part of their normal daily activity. It also alleviates the need for the patient to keep track of the elapsed days in her menstrual cycle, this function being performed by the placebo dosages. Thus, a patient using this method of administering a pharmaceutical for the treatment of one or more symptoms associated with her menstrual cycle, such as the treatment of PMS/PMDD, should have a higher degree of compliance, a higher degree of accurate luteal phase dosing, and therefore a higher likelihood of efficacious dosing than one who is required to count off elapsed days in her menstrual cycle in order to intermittently administer the necessary dosages.

### BRIEF DESCRIPTION OF THE DRAWING

FIGS. 1-5 illustrate exemplary packages of this invention for practicing the method of the invention. FIGS. 6, 7 and 8 illustrate exemplary packages which are preferred embodiments of this invention for practicing the method of the invention.

### DETAILED DESCRIPTION

This invention contemplates the use of any pharmaceutical, i.e., a substance, used in the treatment of a disease, an illness, or the symptoms of a disease, illness or physiological condition associated with a female's menstrual cycle. Exemplary pharmaceuticals employed in the present invention include selective serotonin reuptake inhibitors, non-steroidal anti-inflammatory drugs (NSAIDs), serotonin 5HT-1 antagonists, retinoids, antibiotics, analgesics, diuretics, and corticosteroids. Fluoxetine is a particularly preferred selective serotonin uptake inhibitor.

Exemplary conditions which may be associated with a female's menstrual cycle include premenstrual syndrome, migraine headache, endometriosis, psoriasis, acne, dysmenorrhea, neurosia, asthma and premenstrual cramps.

As used herein, the phrase “daily placebo dosage” refers to the entire amount of the placebo to be administered to the patient in one day. The entire amount may be in a single dosage unit, or may be divided into a number of separate units to be administered at different time periods during the course of the day.

As used herein, the phrase “daily pharmaceutical dosage” refers to the entire amount of a pharmaceutical to be administered to the patient in one day. The entire amount may be in a single dosage unit, or may be divided into a number of separate units to be administered at different time periods during the course of the day.

The phrase “first dosage” as used herein refers to either the pharmaceutical dosages or the placebo dosages, whichever are administered initially from the package. The phrase “first dosage” also refers to the entire dosing regimen for that type of dosage, for example, if the daily pharmaceutical dosages are administered initially, then the phrase “first dosage” refers to all of the daily pharmaceutical dosages to be administered during the menstrual cycle prior to the administration of the daily placebo dosages, which in this example would be the second dosage.

The phrase “second dosage” as used herein refers to the total amount of the daily dosages, either pharmaceutical or placebo, that are to be administered after the administration of the first dosage has been completed.

As used herein, the term “fluoxetine” refers to the following compound: 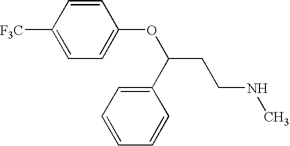
or a pharmaceutically acceptable salt thereof. The specific dose level of fluoxetine for any particular patient will depend upon a variety of factors, including the patient's age, body weight and the severity of the symptoms. Typically this amount would be between about 0.01 to about 300 mg/kg of body weight, with a preferred amount being between about 5 to about 60 mg/day more preferably about 10 to about 20 mg/day. A skilled artisan could routinely determine the proper amount for an individual patient.

As used herein, the phrase “pharmaceutically acceptable salt” refers to a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, g-hydroxybutyrates, glycollates, tartrates, methane-sulfonates (mesylates), propanesulfonates, naphthalene-1-sulfonates, naphthalene-2-sulfonates, and mandelates. A desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid, or the like. In the present invention the hydrochloride salt is the preferred salt.

As used herein, the term “placebo” refers to a dosage form that does not contain a therapeutically effective amount of a pharmaceutical. The placebo could contain either one or more vitamins and, or, minerals. Preferably the placebo is an inert medicament which has no therapeutic value. In the present invention the placebo dosages are administered to the patient on days when the pharmaceutical is not required to be administered to treat the symptoms associated with a female's menstrual cycle, such as PMS. The effect is that the patient must administer a dosage, of either the pharmaceutical or a placebo, every day. The repetition of daily administration will eventually become part of the patient's every day routine, thereby increasing the likelihood of compliance. The placebo dosages in the present invention also eliminate the need for the patient to keep track of the elapsed days after the first day of her menstrual cycle.

As used herein, the phrase “single dosage unit” is used to indicate that the entire daily dosage is contained in one tablet, capsule (e.g., “PULVULES” a registered trademark of Eli Lilly and Company), caplet, or any other vehicle commonly known by those skilled in the art.

Ascertaining the number of days in the female's menstrual cycle is generally not complex. For example, the number of days in a female's menstrual cycle is often known by the female. If not, it can be readily determined by simply counting the days between the onset of menses. For some females, the menstrual cycle varies in length. A menstrual cycle can vary in length between 24 to 33 days in 95% of women with regular cycles, wherein the average length is 28 days. In that case, the usual number of days in the female's menstrual cycle may be used.

In a particularly preferred embodiment of the method of the present invention, the first dosage is the placebo and the second dosage is the pharmaceutical. It is also possible, however, for the first dosage to be the pharmaceutical and the second dosage to be the placebo. In one embodiment of the invention, administration of the first dosage is started on the first day of the menstrual cycle, i.e., the first day of menstruation. In an alternate embodiment, administration of the first dosage is started on the second day of the menstrual cycle, i.e., the day after menstruation begins.

As noted previously, when the first dosage is the placebo and the second dosage is the pharmaceutical, the second dosage period is the selected number of days to administer the pharmaceutical. This period is selected to achieve effective treatment of the condition that is being treated. The first dosage period is then determined by substracting the number of treatment days from the number of ascertained days in the female's menstrual cycle. A similar calculation is made when the first dosage is the pharmaceutical and the second dosage is the placebo, except that it is the number of days of administering the first dosage which is subtracted from the ascertained days in the female's menstrual cycle. In either embodiment, it is the number of days of administration of the pharmaceutical selected to achieve effective treatment of the condition being treated that is subtracted from the ascertained number of days of the female's menstrual cycle to determine the number of days of placebo administration.

As previously indicated, when the second dosage is a pharmaceutical, it may be preferable upon completion of the second dosage period to continue the daily oral administration of the second daily dosage until the first of, exhaustion of the pharmaceutical in the package or the start of menses. This optional embodiment may be useful when the human female's menstrual cycle exceeds the ascertained number of days in the female's menstrual cycle. It should be apparent in this case that the number of days that the pharmaceutical will be administered may exceed the second dosage period, i.e., the number of days selected to achieve effective treatment of the condition that is being treated. For example, while the selected number of days to achieve effective treatment for a given pharmaceutical might be 15 days, a package could contain a greater number of daily dosages of the pharmaceutical, e.g. 18 days, to provide for additional daily dosages of the pharmaceutical for those instances when the female's menstrual cycle exceeded the usual number of days ascertained for her menstrual cycle.

The days of administration of the pharmaceutical could be as many as 25 days to as little as 1 day per cycle. Typically the pharmaceutical is administered using the method of the invention for 5 to 21 days, more preferably for 5 to 15 days and even more preferably 15 days with additional daily dosages up to 20 days if the human female exceeds her ascertained menstrual cycle. It is important to note that the placebos and pharmaceutical dosages used in the present invention are provided in a single package.

In a more specific embodiment of the method of the present invention, the first dosage is the placebo and the second dosage is the pharmaceutical, and the preferred condition of treatment is premenstrual syndrome. In a more preferred embodiment of this method the pharmaceutical is a selective serotonin reuptake inhibitor. A more specific preferred embodiment is where the selective serotonin reuptake inhibitor is selected from the group consisting of citalopram, escitalopram, fluvoxamine, paroxetine, fluoxetine, sertraline, duloxetine and pharmaceutically acceptable salts thereof. In the most preferred embodiment the selective serotonin reuptake inhibitor is fluoxetine or a pharmaceutically acceptable salt thereof.

This method of treating PMS with fluoxetine differs from treatment with oral contraceptives. Both oral contraceptives and the present invention may be packaged in calendar or dial packs. Oral contraceptives, however, entrain the menstrual cycle, and therefore, do not have to contend with cycle lengths other than 28 days. Where as the present invention is designed to accommodate any women's menstrual cycle, regardless of the length.

The present invention also allows for at least a 50% dosage reduction in the average woman with PMS compared to a continuous dosing regimen. Thus, the present pharmaceutical regimen not only allows for ease of compliance, but may be safer.

In another specific embodiment of the method of this invention the selected number of days to administer fluoxetine, or a pharmaceutically acceptable salt thereof, during the second dosage period is in a range of 5 to 14 days. Even more preferred is where the second dosage period is 14 days. In this embodiment the first dosage period is generally determined to be a number in a range of 12 to 20 days. Preferably the package contains 14 daily dosages of fluoxetine, or a pharmaceutically acceptable salt thereof, and about 20 daily dosages of placebo.

In yet another preferred embodiment of this invention, the daily first dosage is a placebo and the daily second dosage is fluoxetine, or a pharmaceutically acceptable salt thereof, and the selected number of days for the second dosage period is in a range of 5 to 15 days. Even more preferred is where the second dosage period is 15 days. In this embodiment the first dosage period is generally determined to be a number in a range of 12 to 20 days. Preferably the package used to practice this embodiment of the method of the invention will contain 15 daily dosages of fluoxetine, or a pharmaceutically acceptable salt thereof and about 19 daily dosages of placebo.

In yet another preferred embodiment, while the second dosage period is 15 days, the package actually may contain up to 20 daily dosages of fluoxetine, or a pharmaceutically acceptable salt thereof, and 12 to 20 daily dosages of placebo, and even more preferably about 17 daily dosages of placebo. In this embodiment it should be apparent that the 15 day second dosage period is used to calculate the number of daily placebo dosages that will be administered based on the ascertained number of days in the female's menstrual cycle. If the onset of menses occurs before all of the daily dosages of fluoxetine, or pharmaceutically acceptable salt thereof, in the package are used, then the female will discard that package and repeat the treatment method by ascertaining her menstrual cycle length and beginning the administration of a placebo from a new package. If the female exceeds her usual menstrual cycle she may take the additional daily dosages of fluoxetine, or pharmaceutically acceptable salts thereof, contained in the package until either the additional dosages are exhausted or menses begins.

Most preferred is where the package of this invention, whether the first dosage is started on the first day or the second day of the menstrual cycle, includes indicia to determine the first dosage period based on the number of days in the female's menstrual cycle. This embodiment may be used to treat premenstrual syndrome, including its more severe form, i.e., premenstrual dysphoric disorder.

This invention is also directed to a package for a non-continuous administration of a pharmaceutical to a human female for a condition associated with the female's menstrual cycle in accordance with the method of this invention. The package comprises an allotment of daily placebo dosages, an allotment of daily pharmaceutical dosages, and a means of determining how many placebo dosage must be administered during the present menstrual cycle. The package may take any shape or form so long as a means is provided to determine the number of placebos that will be administered during the female's menstrual cycle. Exemplary means for determining the number of daily placebo dosages to be administered during the present menstrual cycle include selection indicia, a dial mechanism or removing a certain number of daily placebo dosages before starting administration from the package.

In another preferred specific embodiment of the package of the present invention the daily placebo dosages and the daily pharmaceutical dosages are single dosage units. In a more specific embodiment the single dosage unit is in a form selected from a tablet, a capsule and a pulvule. In a more preferable specific embodiment, wherein the pharmaceutical dosage is fluoxetine, or a pharmaceutically acceptable salt thereof, the single dosage unit is in pulvule form.

As noted previously, the package includes a means for determining how many daily placebo dosages should be administered during the female's menstrual cycle. The means could be indicia on the package that indicates which dosage the patient should begin administration with based upon her usual menstrual cycle. Another means is where the patient moves or slides a sheet or a dial within the package to indicate the normal menstrual cycle length and the movement of the sheet or the dial would also move an indicia that indicates which dosage the patient should start with. The means could also be directions in or on the package instructing the patient to remove a certain amount of placebo dosages prior to starting the administration from that package. Other means of identifying the date on which a dosage unit is consumed will be readily apparent to those skilled in the art.

In a preferred embodiment the package of this invention will contain an allotment of daily placebo dosage that will be used as the first daily dosage and an allotment of daily pharmaceutical dosage that will be used as the second daily dosage to practice the method of this invention. Most preferably the allotment of daily pharmaceutical dosage will include fluoxetine or a pharmaceutically acceptable salt thereof. In a particularly preferred embodiment the package will be a card design having the allotment of daily placebo and daily pharmaceutical dosage disposed thereon. Preferably each dosage may be individually removed from the package such as in a blister pack type package. In a particularly preferred embodiment the selection indicia on the package will be sequential numbers with each number being associated with a daily dosage. Most preferably the highest number will be associated with a daily placebo dosage and count down through the rest of the placebo dosages, most preferably to the number sixteen. Then in this preferred embodiment the number will continue from fifteen to one through the daily pharmaceutical dosages. Instructions either on the package or accompanying this preferred package may instruct a female to ascertain the usual number of days in her menstrual cycle and to begin administration of the daily placebo dosage associated with the number that matches her usual number of days in her menstrual cycle. The user may also be instructed to continue daily administration of each dosage in sequential order until the first of either the pharmaceutical dosages are exhausted or the start of menses. In that regard it should be noted that the packages may be designed to include extra pharmaceutical dosages with instructions to administer those extra dosages if the first fifteen pharmaceutical dosages are completed prior to the onset of menses. The user is preferably instructed to discard the package once menses begins and start a new package. However, prior to starting a new package, in a preferred embodiment, the package may contain instructions that indicate that the user should ascertain her usual menstrual cycle based on the last package used. For example, instructions may be provided that ask the user to subtract the number associated with the pharmaceutical tablets that remained in the package just finished (not counting any extra pharmaceutical dosage described above) from the number that was used to start the last package to ascertain the number to be used to start the new package.

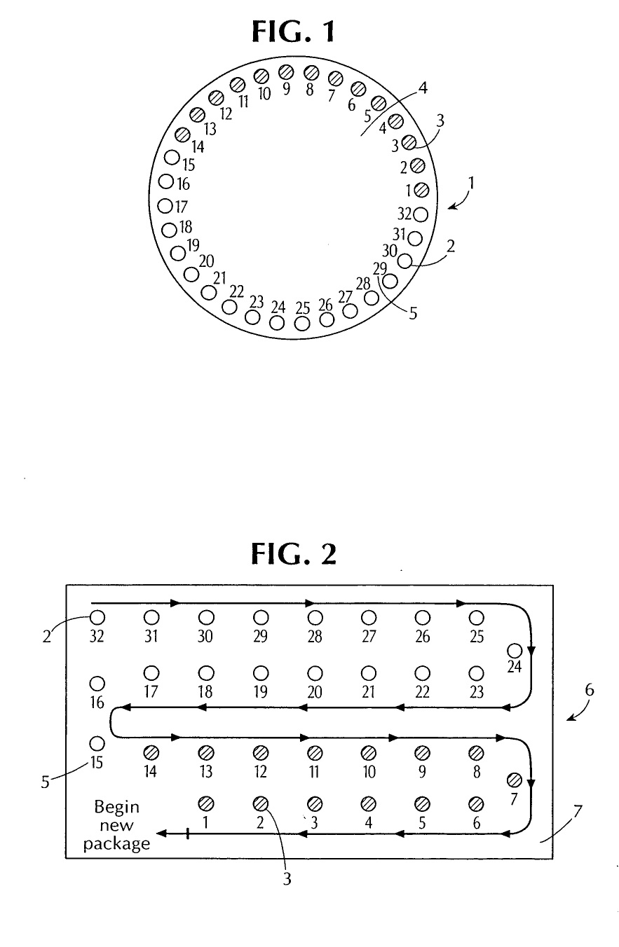

FIG. 1 is an illustration of exemplary package 1 of the present invention. The package includes an allotment of eighteen daily placebo dosages 2 and an allotment of fourteen daily pharmaceutical dosages 3. Both the placebo and pharmaceutical dosages are disposed on a circular base card 4, where they are held by any suitable means, e.g. blister packaging. The base card includes numbers 5 corresponding to the length of the female's menstrual cycle. A female would start administration with the placebo dosage that corresponds to the usual number of days in her menstrual cycle, taking one dosage daily, counting down on the package to the next lowest number each day. Once the last pharmaceutical dosage is administered the package is discarded and administration begins from a new package, or, if a new menstrual cycle begins before the last pharmaceutical dosage is administered, then the package is discarded and administration begins from a new package.

FIG. 2 is an illustration of exemplary package 6 of the present invention. The package includes a total of thirty two dosages, wherein eighteen are daily placebo dosages 2 and fourteen are daily pharmaceutical dosages 3. Both the placebo and pharmaceutical dosages are disposed on a rectangular base card 7, where they are held by any suitable means, e.g. blister packaging. The base card includes numbers 5 corresponding to the length of the female's menstrual cycle. A female would start administration with the placebo dosage that corresponds to the usual number of days in her menstrual cycle, taking one dosage daily, counting down on the package to the next lowest number each day. Once the last pharmaceutical dosage is administered the package is discarded and administration begins from a new package, or, if a new menstrual cycle begins before the last pharmaceutical dosage is administered, then the package is discarded and administration begins from a new package.

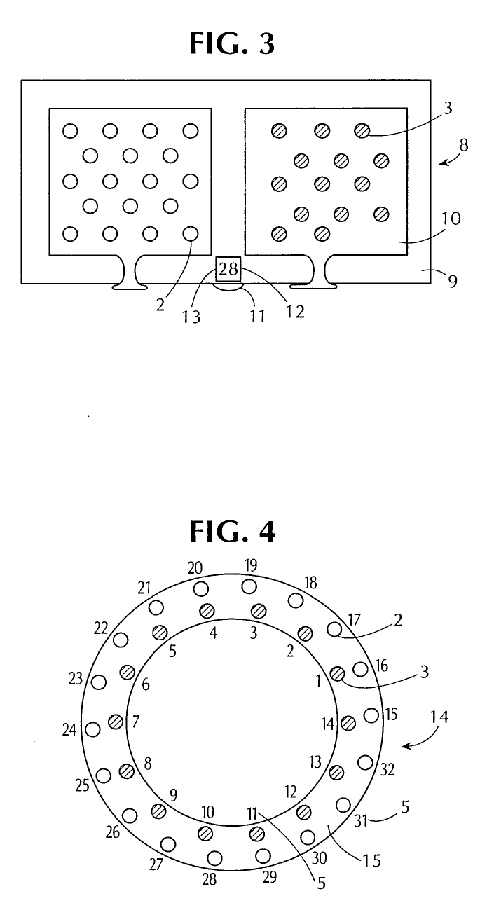

FIG. 3 is an illustration of exemplary package 8 of the present invention. The package includes a total of thirty two dosages, wherein eighteen are daily placebo dosages 2 and fourteen are daily pharmaceutical dosages 3. Rectangular base card 9 contains two reservoirs 10. One reservoir contains all of the placebo dosages, while the other reservoir contains all of the pharmaceutical dosages. The dosages are held within the reservoirs by any suitable means, e.g. blister packaging. A dial 11 having an indicator slot 12 is rotatably mounted on the base card. The dial has numbers 13 from one to thirty two on it, such that the numbers can be seen through the indicator slot. A female would begin administration from this package on the first day of menses by turning the dial till the number corresponding to the usual length, in days, of her menstrual cycles is visible through the indicator slot. Then the female would administer one placebo dosage daily and turn the dial so that the next lowest number will appear through the indicator slot. This would be repeated daily until the number fourteen is visible through the indicator slot. Once fourteen is visible, daily administration of one pharmaceutical dosage would begin and the dial would continue to be turned so that the next lowest number is visible through the indicator slot. When the last pharmaceutical dosage is administered the package is discarded and administration is begun again from a new package, or, if a new menstrual cycle has begun before the last pharmaceutical dosage is administered, then the package is discarded and administration is begun from a new package.

FIG. 4 is an illustration of exemplary package 14 of the present invention. The package includes an allotment of eighteen daily placebo dosages 2 and an allotment of fourteen daily pharmaceutical dosages 3. Both the placebo and pharmaceutical dosages are disposed on a circular base card 15, where they are held by any suitable means, e.g. blister packaging. The placebo dosages are disposed to form an outer ring and the pharmaceutical dosages are disposed to form an inner ring. The base card includes numbers 5 corresponding to the length of the female's menstrual cycle. A female would start administration with the placebo dosage that corresponds to the usual number of days in her menstrual cycle, taking one dosage daily, counting down on the package to the next lowest number each day. Once the last pharmaceutical dosage is administered the package is discarded and administration begins from a new package, or, if a new menstrual cycle begins before the last pharmaceutical dosage is administered, then the package is discarded and administration begins from a new package.

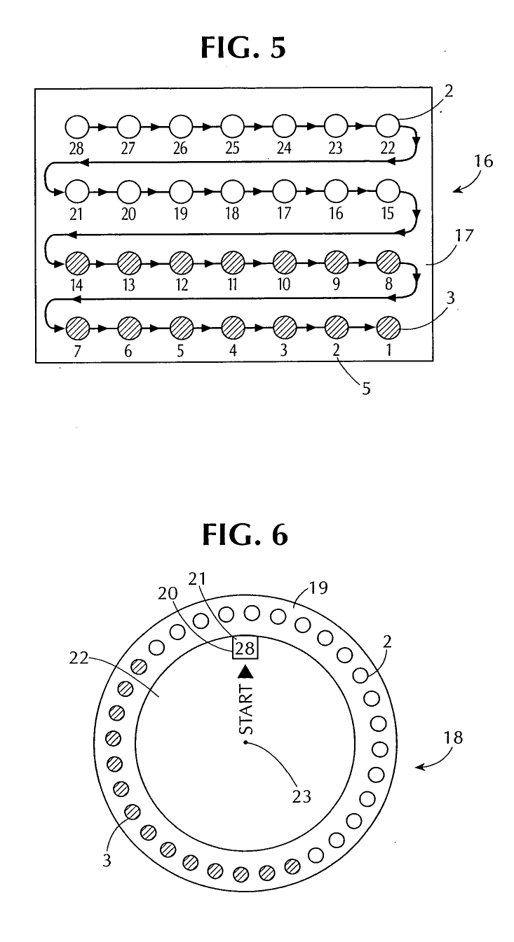

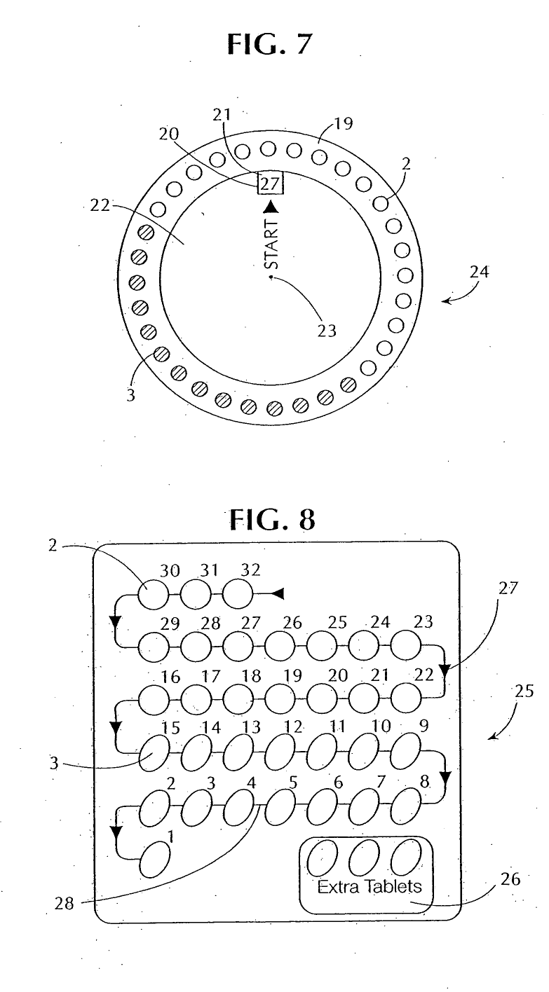

FIG. 5 is an illustration of exemplary package 16 of the present invention. The package includes a total of twenty eight dosages, wherein fourteen are daily placebo dosages 2 and fourteen are daily pharmaceutical dosages 3. Both the placebo and pharmaceutical dosages are disposed on a rectangular base card 17, where they are held by any suitable means, e.g. blister packaging. The base card includes numbers 5 corresponding to the length of the female's menstrual cycle. A female would start administration with the placebo dosage that corresponds to the usual number of days in her menstrual cycle, taking one dosage daily, counting down on the package to the next lowest number each day. Once the last pharmaceutical dosage is administered the package is discarded and administration begins from a new package, or, if a new menstrual cycle begins before the last pharmaceutical dosage is administered, then the package is discarded and administration begins from a new package.

FIG. 6 is an illustration of exemplary package 18 of the present invention. The package includes an allotment of eighteen daily placebo dosages 2 and an allotment of fourteen daily pharmaceutical dosages 3. Both the placebo and pharmaceutical dosages are disposed on a circular base card 19, where they are held by any suitable means, e.g. blister packaging. A dial card 22 having an indicator slot 20 is rotatably mounted on the base card by rivet 23. The base card includes numbers 21 corresponding to the length of the female's menstrual cycle so that when the numbers show through the indicator slot 20 then the appropriate number of daily placebo dosages will be administered to ensure that the female receives substantially all of her allotment of pharmaceutical dosages before her next menses. In the examplary embodiment, 32 dosages are provided on the package, and the indicator is set to administer placebo and pharmaceutical dosages for a female that has ascertained that her menstrual cycle is 28 days in length. When the last pharmaceutical dosage is administered the package is discarded and administration begins from a new package, or, if a new menstrual cycle begins before the last pharmaceutical dosage is administered, then the package is discarded and administration begins from a new package.

The packages illustrated in FIGS. 1-6 could readily be altered to hold different amounts of the pharmaceutical dosage and the placebo dosage. Similarly, instructions and/or indicia on the packages could readily be altered for administration of the placebo dosage to begin on the second day of the menstrual cycle. For example, the packages illustrated in FIGS. 1-4 could be readily altered to include fifteen daily pharmaceutical dosages 3 and seventeen daily placebo dosages 2. These altered packages could include instructions to begin administration of the placebo dosage on the first or the second day of the menstrual cycle, as desired, starting with the placebo dosage corresponding to the number of days remaining in the menstrual cycle. For example, in the package illustrated in FIG. 3, the female would be instructed to set the dial to the usual number of days in the menstrual cycle and the daily administration of the pharmaceutical dosage would begin when the dial indicator showed fifteen through the indicator slot. In the most preferred embodiment of this invention, using these packages, if the female's menstrual cycle does not vary, then the last daily dosage of pharmaceutical dosage would be administered on the last day of the females menstrual cycle. The next day's administration would be started from a new package. In this embodiment, if the package still contained pharmaceutical dosage after the first day of the menstrual cycle, the package would be discarded and administration would begin from a new package.

The illustrated in FIG. 7 could be used to start administration of the placebo dosage on the first or second day of the menstrual cycle. Package 24 is similar to that shown in FIG. 6, except that it contains fifteen daily pharmaceutical dosages 3 and seventeen daily placebo dosages 2. In addition, the package 24 includes indicia and/or instructions indicating that the administration of the placebo dosage 2 should be begun. If it were desired to have administration begin on the second day of the menstrual cycle then the female would be instructed to set the numbers 21 displayed in indicator slot 20 to the days remaining in her menstrual cycle. For example, if the female's usual menstrual cycle is 28 days in length then on the second day of the menstrual cycle the dial card 22 is rotated so that the number twenty seven, i.e., the days remaining in the menstrual cycle, is displayed in the indicator slot 20. Thereafter, a dosage is administered daily moving in a clockwise direction around circular base card 19. If it were desired to have administration begin on the first day of menses then the female would be instructed to set the numbers 21 displayed in indicator slot 20 to the ascertained days in her menstrual cycle. For example, if the female's usual menstrual cycle were 21 days in length then on the first day of her menstrual cycle the dial card 22 would be rotated to that number. When the last pharmaceutical dosage is administered the package is discarded and administration begins from a new package, or, if a new menstrual cycle is reached before the last pharmaceutical dosage is administered, then the package is discarded and administration begins from a new package.

It is also possible, of course, for these packages illustrated in FIGS. 1-7 to contain more than 15 daily pharmaceutical dosages, even if 15 daily dosages is considered the desired second dosage period. Those embodiments would then provide additional daily pharmaceutical dosages for these instances when the length of the female's menstrual cycle exceeded the ascertained days of the female's menstrual cycle. In such an embodiment, using a package having an indicator slot such as illustrated in FIG. 3, 6 or 7, it might be preferable for the indicator to show an indication that the user has reached the additional daily pharmaceutical dosages. This could be done, for example, by using numbers of a different color and starting to count up from one. So for example, if a female's menstrual cycle exceeded her usual cycle by three days, then on the second day past her usual cycle the indicator would have the number two showing in a color different than that used for the numbers during the usual menstrual cycle or some other indicia to show that additional daily dosages were being administered.

A particularly preferred embodiment of this invention is illustrated in FIG. 8. Package 25 is a card containing 15 pharmaceutical dosages 3 in numbered order and 17 placebo dosages 2 in numbered order. The package 25 also contains three extra pharmaceutical dosages 3 in a separate area 26. The card includes numbers 5 from thirty two to one which are associated with the placebo dosages 2 and the pharmaceutical dosages 3. The package preferably includes instructions, which may be written on the package and/or included on a package insert, that indicate that a female should ascertain the usual number of days in her menstrual cycle and that administration of placebo dosage 2 should start on the first day of menses at the number 5 that corresponds to the ascertained length of her menstrual cycle. Thereafter, administration proceeds on a daily basis to the dosage associated with the next lowest number in the direction of the arrows 27 following pathway 28. When the last pharmaceutical dosage 3 along pathway 28 is administered the user may optionally begin administration of the pharmaceutical dosage 3 contained in the extra dosage area 26 until either the pharmaceutical dosage 3 in the extra dosage area 26 is exhausted or the onset of menses occurs. When either the last pharmaceutical dosage is administered, or if a new menstrual cycle begins before the last pharmaceutical dosage is used then the package is discarded. Administration from a new package may begin when a new menstrual cycle starts.

Of course, alternate embodiments of all of these packages can be readily envisioned.

1. A method of non-continuous administration of a pharmaceutical to a human female for a condition associated with the female's menstrual cycle, comprising the steps of:
a) ascertaining a number of days in the female's menstrual cycle;
b) orally administering a daily first dosage, starting on the first day of the female's menstrual cycle and continuing the daily oral administrations of said first dosage for a first dosage period; and
c) orally administering a daily second dosage, starting on the day after the last daily first dosage was administered and continuing the daily oral administration of said second dosage for a second dosage period or until the onset of menses;
wherein either (i) said first dosage is a placebo and said second dosage is said pharmaceutical, and said second dosage period is a selected number of days to administer said pharmaceutical for effective treatment of said condition and said first dosage period is determined by subtracting the selected number of days from the number of days in the female's menstrual cycle; or (ii) said first dosage is said pharmaceutical and said second dosage is a placebo, and said first dosage period is a selected number of days to administer said pharmaceutical for effective treatment of said condition and said second dosage period is determined by subtracting the selected number of days from the number of days in the female's menstrual cycle; and
wherein the first dosage and the second dosage administered during said menstrual cycle are selected from a single package.

2. The method of claim 1, wherein said first dosage is said placebo and said second dosage is said pharmaceutical.

3. The method of claim 1, wherein said first dosage is said pharmaceutical and said second dosage is said placebo.

4. The method of claim 1, wherein said condition is selected from the group consisting of premenstrual syndrome, migraine headache, endometriosis, psoriasis, acne, dysmenorrhea, neurosia, and premenstrual cramps.

5. The method of claim 2, wherein said condition is premenstrual syndrome.

6. The method of claim 5, wherein said pharmaceutical is a selective serotonin reuptake inhibitor.

7. The method of claim 6, wherein said selective serotonin reuptake inhibitor is selected from the group consisting of citalopram, escitalopram, fluvoxamine, paroxetine, fluoxetine, sertraline and pharmaceutically acceptable salts thereof.

8. The method of claim 7, wherein said selective serotonin reuptake inhibitor is fluoxetine or a pharmaceutically acceptable salt thereof.

9. The method of claim 8, wherein the selected number of days to administer said fluoxetine, or the pharmaceutically acceptable salt thereof, during the second dosage period is in a range of 5 to 21 days.

10. The method of claim 9, wherein said second dosage period is 14 days.

11. The method of claim 9, wherein said second dosage period is 15 days.

12. The method of claim 11, wherein the first dosage period is determined to be a number in a range of 12 to 20 days.

13. The method of claim 12, wherein said package contains 15 daily dosages of fluoxetine, or the pharmaceutically acceptable salt thereof and about 12 to 20 daily dosages of placebo.

14. The method of claim 13, wherein said package includes indicia to determine the first dosage period based on the number of days in the female's menstrual cycle.

15. The method of claim 12, wherein said package contains 15 to 20 daily dosages of fluoxetine, or the pharmaceutically acceptable salt thereof and said method further comprises the step of orally administering said daily dosages of fluoxetine, or the pharmaceutically acceptable salt thereof, until the first of either the pharmaceutical dosages in said package are exhausted or the onset of menses of the female.

16. The method of claim 15, wherein said package contains about 17 to 19 daily dosages of placebo.

17. The method of claim 16, wherein said package includes indicia to determine the first dosage period based on the number of days in the female's menstrual cycle.

18. The method of claim 5, wherein said premenstrual syndrome is premenstrual dysphoric disorder.

19. The method of claim 1, wherein each first dosage and second dosage is contained in a single dosage unit.

20. The method of claim 1, wherein said pharmaceutical is selected from the group consisting of a selective serotonin reuptake inhibitor, an analgesic, a diuretic, and cortico steroids.

21. The method of claim 1, wherein the selected number of days to administer said pharmaceutical is in a range of 5 to 21 days.

22. The method of claim 1, wherein steps a, b and c are repeated at the onset of menses.

23. A package for delivering a non-continuous administration of a pharmaceutical to a human female for a condition associated with the female's menstrual cycle, said package comprising:
an allotment of daily placebo dosages;
an allotment of daily pharmaceutical dosages; and
a means of determining how many daily placebo dosages must be administered during the female's menstrual cycle.

24. The package of claim 23, wherein the means of determining the number of daily placebo dosages to be administered during the present menstrual cycle is selected from the group consisting of selection indicia, a dial mechanism and removing a certain number of daily placebo dosages prior to starting administration from said package.

25. The package of claim 24, wherein said condition is selected from the group consisting of premenstrual syndrome, migraine headache, endometriosis, psoriasis, acne, dysmenorrhea, neurosia, asthma and premenstrual cramps.

26. The package of claim 25, wherein said condition is premenstrual syndrome.

27. The package of claim 26, wherein said daily pharmaceutical dosages include a selective serotonin reuptake inhibitor.

28. The package of claim 27, wherein said selective serotonin reuptake inhibitor is selected from the group consisting of citalopram, escitalopram, fluvoxamine, paroxetine, fluoxetine, setraline, duloxetine and pharmaceutically acceptable salts thereof.

29. The package of claim 28, wherein said selective serotonin reuptake inhibitor is fluoxetine or a pharmaceutically acceptable salt thereof.

30. The package of claim 29, wherein the number of daily pharmaceutical dosages is in a range of 5 to 21.

31. The package of claim 30, wherein the number of daily pharmaceutical dosages is about 15.

32. The package of claim 31, wherein there are about 12 to 20 daily placebo dosages.

33. The package of claim 32, wherein said daily placebo dosages and said daily pharmaceutical dosages are each single dosage units.

34. The package of claim 33, wherein the single dosage units are in a form selected from the group of a tablet, caplet and a capsule.

35. The package of claim 30, wherein the number of daily pharmaceutical dosages is in a range of 15 to 20.

36. The package of claim 35, wherein there are about 12 to 20 daily placebo dosages.

37. The package of claim 36, wherein there are about 17 to 19 daily placebo dosages.

38. The package of claim 37, wherein said daily placebo dosages and said daily pharmaceutical dosages are each single dosage units.

39. The package of claim 38, wherein the single dosage units are in a form selected from the group of a tablet, caplet and a capsule.

40. The package of claim 26, wherein said premenstrual syndrome is premenstrual dysphoric disorder.
